(19) United States
(12) Patent Application Publication
(10) Pub. No.: US 2023/0405269 A1
(43) Pub. Date: Dec. 21, 2023

(54) SINGLE-CATHETER ENABLED PDA ACCESS AND OCCLUDER DEPLOYMENT AND RECAPTURE

(71) Applicant: St. Jude Medical, Cardiology Division, Inc., St. Paul, MN (US)

(72) Inventors: Brian Perszyk, Shoreview, MN (US); Erika Beek, Bloomington, MN (US); Alan Twomey, Minneapolis, MN (US)

(21) Appl. No.: 18/338,557

(22) Filed: Jun. 21, 2023

Related U.S. Application Data
(60) Provisional application No. 63/354,217, filed on Jun. 21, 2022.
(60) Provisional application No. 63/494,373, filed on Apr. 5, 2023.

(51) Int. Cl. A61M 25/00; A61M 25/01

(52) U.S. Cl. CPC A61M 25/005; A61M 25/0108; A61M 25/0045; A61M 2025/0046; A61M 2025/0008

(57) ABSTRACT

The present disclosure describes a catheter including a shaft formed from braided material encapsulated by a resin jacket. The catheter shaft extends from a proximal portion to a distal tip, and includes a distal curved section including an acute distal curve proximal of the distal tip.

### CROSS-REFERENCE TO RELATED APPLICATIONS

This application claims the benefit of priority to U.S. Provisional Patent Application No. 63/354,217, filed Jun. 21, 2022, and to U.S. Provisional Patent Application No. 63/494,373, filed Apr. 5, 2023, the entire contents and disclosure of each of which are incorporated by reference herein.

### BACKGROUND OF THE DISCLOSURE

### a. Field of Disclosure

The present disclosure generally relates to a medical device delivery catheter. In particular, the present disclosure relates to a catheter with a distal curved region and having enhanced softness, for improved access to vascular abnormalities, heart abnormalities, tissue defects, and for improved deployment and recapture of medical devices within a patient's body.

### b. Background Art

Delivery devices including, among other components, catheters and delivery cables are used for an ever-growing number of procedures, and, in particular, for the delivery of medical devices to a target site. Typically, the catheter is manipulated through the patient's vasculature and to the intended site, such as, for example, a site within the patient's heart or other organ, and the delivery cable is used to advance the medical device through the catheter and to the target site. Once the medical device has reached the target site, the delivery cable may be detached or uncoupled from the medical device such that the medical device is deployed from both the catheter and the delivery cable.

Generally, the catheter would have an overall outside diameter small enough to navigate blood vessels or other anatomy while retaining an inner diameter (e.g., a bore size) large enough to accommodate the medical device and delivery cable therethrough. Because the path within the patient may be long, tortuous, and/or involve intricate placement of a medical device(s), maneuverability via steering the catheter may be particularly beneficial. Furthermore, the delivery cable must be rigid enough to be capable of maneuvering the medical device through the catheter while still being flexible enough to accommodate the tortuous path through which it must travel to the target site.

In some specific instances, such as for cardiac procedures performed in particularly young and/or small patients, these abovementioned concerns are enhanced. In particular, the delivery catheter should be small enough, have enough curvature, and be soft enough to traverse the path to the target site (e.g., a patent ductus arteriosus, or PDA). In some cases, a first guide catheter—optimized to traverse the vascular system but not to deliver the medical device—is used to position a guidewire relative to the target site. Then, the first guide catheter is removed and exchanged for a second catheter suitable to deliver the medical device to the target site. However, it would be desirable to eliminate this catheter exchange procedure, to reduce the potential for damaging tissue at the target site and/or along the vascular path thereto.

In other cases, a smaller guidewire is used to guide a delivery catheter to the target site for deployment of the medical device. However, in these cases, the diameter of the guidewire is significantly smaller than an inner diameter of the delivery catheter. This mismatch between the size of the guidewire and the inner diameter of the delivery catheter may, in some instances, increase the risk of damage to tissue through the vasculature up to and including the target site, as the space between the catheter and the guidewire exposes a gap that can catch on or entrap tissue, and exposes the edge of the catheter, which can be traumatic to the tissue.

### SUMMARY OF THE DISCLOSURE

The present disclosure is directed to a catheter including a shaft formed from braided material encapsulated by a resin jacket, wherein the catheter shaft extends from a proximal portion to a distal tip, the catheter shaft including a distal curved section including an acute distal curve proximal of the distal tip.

The catheter may be a 3-5 French (Fr) catheter.

The resin jacket may have a varying durometer (hardness) along a length thereof. The resin jacket may have a lower durometer along the distal curved section and a higher durometer proximal of the distal curved section.

The distal tip may be rounded, thick, blunt, and/or soft, due at least in part to the resin jacket.

A portion of the resin jacket including or adjacent to the distal tip may be echogenic.

The distal curved section may also include a proximal curve proximal of the distal curve. The distal curved section may have a resultant curve angle that directs the distal tip downward and obliquely away from a proximal-to-distal direction of a longitudinal axis of the shaft. The proximal curve may have a larger bend radius and arc length than a bend radius and arc length of the acute distal curve. The distal curved section may further include a second-proximal curve proximal of the proximal curve. The second-proximal curve may be spaced from the proximal curve by a straight section.

The catheter shaft may have an inner diameter that decreases towards the distal tip.

The catheter shaft may further include a coil section distal of the braided material at the distal tip.

The catheter may further include (i) a hydrophilic coating on the resin jacket, or (ii) a lubricious additive in the resin jacket.

The catheter may further include a radiopaque markerband adjacent to the distal tip.

A portion of the resin jacket including or adjacent to the distal tip may include a radiopaque material additive that acts as a radiopaque marker.

The catheter may further include a hub adjacent to the proximal portion of the catheter shaft. The hub may include a visual indicator aligned with a direction of the distal curve.

The catheter may further include a marker on the proximal portion thereof.

In another aspect of the present disclosure, a medical device delivery and recapture system may include a dilator and a catheter including a shaft formed from braided material encapsulated by a resin jacket, wherein the catheter shaft extends from a proximal portion to a distal tip, the catheter shaft including a distal curved section including an acute distal curve proximal of the distal tip.

The foregoing and other aspects, features, details, utilities, and advantages of the present disclosure will be apparent from reading the following description and claims, and from reviewing the accompanying drawings.

### BRIEF DESCRIPTION OF THE DRAWINGS

FIG. 1 is a perspective view of one embodiment of a catheter assembly in accordance with the present disclosure;

FIG. 2 is a schematic diagram of a portion of a shaft of the catheter assembly shown in FIG. 1;

FIG. 3 is a schematic diagram illustrating characteristics of a distal curved region of the catheter shaft, including a primary or distal curve;

FIGS. 4A-4C depict various embodiments of the distal curved region of the catheter shaft, including the distal curve;

FIG. 5 is a schematic diagram illustrating characteristics of the distal curved region of the catheter shaft, including a secondary or proximal curve as well as the distal curve;

FIGS. 6A-6D depict various embodiments of the distal curved region of the catheter shaft, including the proximal and distal curves;

FIG. 7 is a schematic diagram illustrating characteristics of the distal curved region of the catheter shaft, including the proximal curve and a second-proximal curve, as well as the distal curve;

FIG. 8 depicts an exemplary embodiment of a curve plate used to form a catheter including a distal curved region having one or more curves and the corresponding catheter;

FIG. 9 depicts another exemplary embodiment of a curve plate used to form a catheter including a distal curved region having one or more curves and the corresponding catheter;

FIG. 10 depicts an exemplary embodiment of a curve plate used to form the distal curved region of a catheter, including a tertiary or “second-proximal” curve, as well as the primary and secondary curves, and the corresponding catheter;

FIG. 11 depicts a further embodiment of a catheter shaft including the distal curved region in accordance with the present disclosure;

FIGS. 12A-12C depict exemplary catheters and illustrate a resultant curve characteristic of the distal curved regions thereof; and

FIGS. 13 and 14 depict embodiments of a proximal portion of the catheter in accordance with the present disclosure, including distance markers.

### DETAILED DESCRIPTION OF THE DISCLOSURE

The present disclosure is directed to a catheter with improved characteristics to traverse a patient's vasculature, while maintaining the functionality to deliver a medical device (e.g., an occluder) to a target site and to maneuver and/or recapture the medical device at the target site.

The disclosed embodiments may lead to more consistent and improved patient outcomes. It is contemplated, however, that the described features and methods of the present disclosure as described herein may be incorporated into any number of systems as would be appreciated by one of ordinary skill in the art based on the disclosure herein.

It is understood that the use of the term “target site” is not meant to be limiting, as delivery systems and/or delivered medical device(s) may be configured to treat any target site, such as an abnormality, a vessel, an organ, an opening, a chamber, a channel, a hole, a cavity, or the like, located anywhere in the body. The term “vascular abnormality,” as used herein, is not meant to be limiting, as the delivery systems and/or delivered medical device(s) may be configured to traverse, bridge, or support a variety of vascular abnormalities. For example, the vascular abnormality could be any abnormality that requires tracking a catheter through the anatomy, and delivery of an occlusion device, such as an atrial septal defect (ASD), a left atrial appendage (LAA), a lesion, a vessel dissection, or a tumor. Embodiments of the medical device may be useful, for example, for occluding a patent ductus arteriosus (PDA), an ASD, a ventricular septal defect (VSD), or an LAA. Furthermore, the term “lumen” is also not meant to be limiting, as the vascular abnormality may reside in a variety of locations within the vasculature, such as a vessel, an artery, a vein, a passageway, an organ, a cavity, or the like. For ease of explanation, the examples used herein refer to the occlusion of a PDA.

As used herein, the term “proximal” refers to a part of the medical device or the delivery system that is closest to the operator, and the term “distal” refers to a part of the medical device or the delivery system that is farther from the operator at any given time as the medical device is being delivered through the delivery device. In addition, the terms “deployed” and “implanted” may be used interchangeably herein.

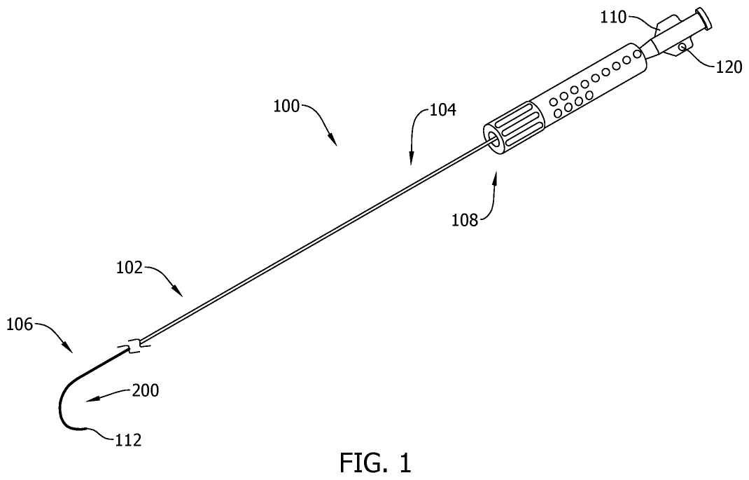

Referring now to the Figures, FIG. 1 is a perspective view of an embodiment of an introducer or catheter assembly 100, including a shaft 102 having a proximal portion 104 and a distal portion 106. Shaft 102 may be operably connected to a handle assembly 108, which assists in guiding or steering shaft 102 during diagnostic, therapeutic, and/or other procedures conducted, for example, in a patient's heart. In other embodiments, catheter assembly 100, also referred to generally as catheter 100, is not coupled to a handle assembly. Catheter assembly 100 may also include a hub 110 operably connected to an inner lumen (not shown) within handle assembly 108 for insertion or delivery of additional medical devices, fluids, or any other devices. FIG. 1 also depicts a distal curved region 200 of distal portion 106. In the exemplary embodiment, catheter 100 is 4 French (4 Fr) catheter suitable for PDA occluder delivery in young or small patients. However, in other embodiments, catheter 100 may be a catheter of a different size, including sizes smaller than 4 Fr, such as about 3 Fr, or larger sizes, such as about 4-5 Fr. In some embodiments, catheter shaft 102 has an effective length between about 43-47 centimeters (cm), or about 45 cm. In other embodiments, catheter shaft 102 may have a shorter effective length (e.g., greater than or equal to 30 cm) or a longer effective length (e.g., less than or equal to 80 cm).

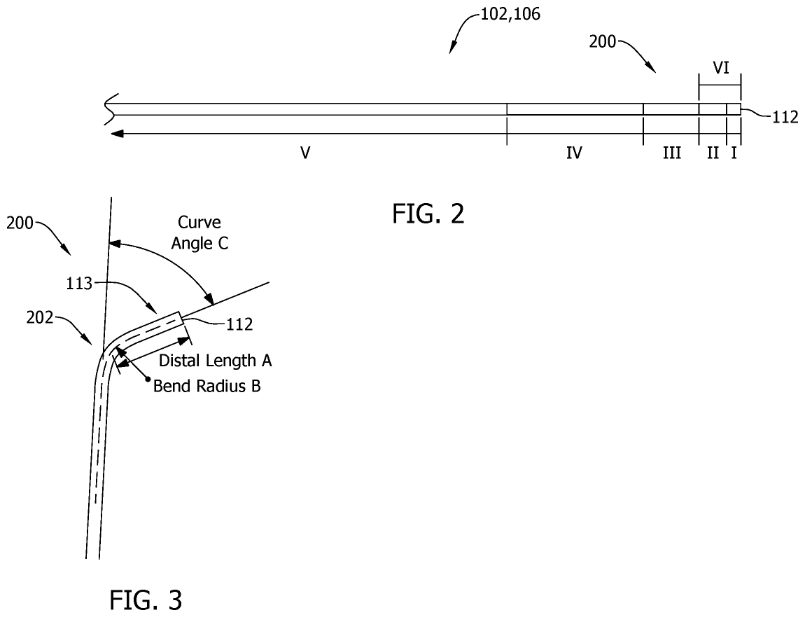

With reference to FIG. 2, a simplified schematic diagram of shaft 102 is illustrated. Distal portion 106 extends proximally from a distal tip 112 (also shown in FIG. 1) and can be said to include sections I-V. Distal curved region 200 may be defined to include, in various embodiments, sections I, I-II, I-III, and/or I-IV. In some embodiments, catheter shaft 102 includes sections I-V, and distal curved region 200 terminates within section III. In other embodiments, catheter shaft 102 includes sections I-V, and distal curved region 200 terminates in section IV. The remainder of shaft 102 proximal of distal portion 106 (e.g., including proximal portion 104) may be said to include section V, which is proximal of section IV. In certain embodiments, proximal portion 104, which is proximal of distal curved region 200, may include portions of sections III and/or IV, depending on where distal curved region 200 terminates, how many sections there are, and the length of the respective sections, as described further herein. Section VI refers generally to the combination of sections I and II. These section labels will be used throughout this disclosure.

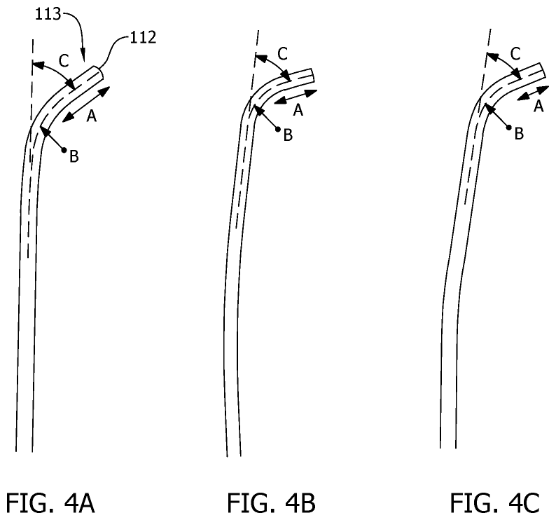

With reference to FIG. 3, in accordance with the present disclosure, distal curved region 200 includes a distal curve 202 proximate to distal tip 112 of catheter shaft 102. Distal curve 202 may be defined to include section I, section II, or section VI (also referred to as sections I-II; see FIG. 2). Distal curve 202 may additionally include at least a portion of section III. FIG. 3 provides definitions for various characteristics or dimensions of distal curve 202 that will be discussed further herein, including a length A, a distal bend radius B, and a curve angle C of distal curve 202. FIGS. 4A-4C depict exemplary embodiments of distal curved region 200 featuring distal curves 202 with varying values of these characteristics.

The length A of distal curve 202 refers to a length of the catheter that extends distally of the physical curve (from the center of distal curve 202) in catheter 100, and extends fully to distal tip 112. The distal bend radius B of distal curve 202 refers to the radius of a hypothetical circle that would be formed from the partial curve or arc that forms the physical curve or bend of distal curve 202 in catheter 100. The curve angle C of distal curve 202 refers to the angle formed between a longitudinal axis extending proximally from the distal tip 112 of catheter 100 through the catheter and a longitudinal axis extending through the portion of catheter 100 immediately proximal to the physical bend that forms distal curve 202, as shown by phantom lines in, for example, FIGS. 4A-4C. Therefore, where distal curve 202 is described as being “acute,” this phrase refers to an angle of less than 90° formed between the longitudinal axis extending proximally from distal tip 112 and the longitudinal axis extending through a portion of catheter 100 proximal to distal curve 202, as illustrated in FIGS. 3 and 4A-4C. A hypothetical catheter 100 having a more acute distal curve 202 (e.g., a smaller acute curve angle C) may be characterized as being relatively straighter than another hypothetical catheter 100 with a less acute distal curve 202 (e.g., with a comparatively larger acute curve angle C). In some embodiments, length A may be about 4 millimeters (mm) to about 14 mm. Therefore, in some embodiments, distal tip 112 is about 8 mm to 9 mm from the center of distal curve 202. Distal bend radius B may be about 1.5 mm to about 1.5 cm. Curve angle C may be about 30° to about 90°, prior to insertion into the anatomy. A distal tip portion 113 extends proximally from distal tip 112 a length of between 0.5 mm to and preferably between 1 mm to 5 mm, and more preferably 1 mm to 3 mm.

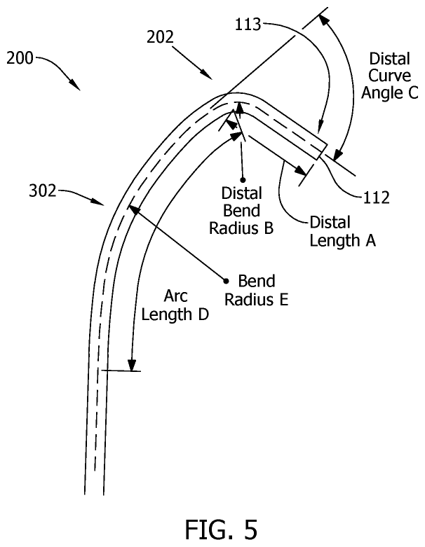

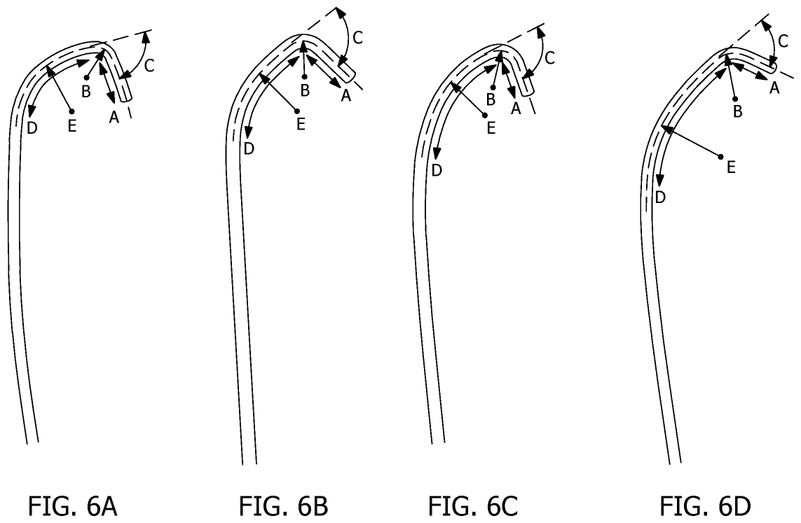

Turning to FIG. 5, another distal curved region 200 is depicted. In this embodiment, distal curved region 200 includes distal curve 202, as well as a secondary curve 302, which is proximal of distal curve 202. In some instances, therefore, secondary curve 302 may be referred to as “proximal” curve 302. Proximal curve 302 may be defined to include all or part of section III, all or part of section IV, or sections and/or all or part of section III and at least part of section IV (see FIG. 2). FIG. 5 provides definitions for various characteristics or dimensions of proximal curve 302 that will be discussed further herein, including an arc length D and a proximal bend radius E. FIG. 5 also illustrates the characteristics (A, B, and C) of distal curve 202, relative to proximal curve 302. Generally, proximal curve 302 has a larger proximal bend radius E than distal curve 202, in that proximal bend radius E is greater than distal bend radius B. FIGS. 6A-6D depict exemplary embodiments of distal curved region 200 featuring distal curves 202 and proximal curves 302 with varying values of their respective characteristics.

In exemplary embodiments, proximal curve 302 commences about to about 3 cm, or about 0 cm to about 1 cm, proximal of distal curve 202. For example, the location of proximal curve 302 may be limited to the section of catheter shaft 102 that, in operation, extends from the right ventricle to the PDA, or may include additional proximal length, such as the section of catheter shaft 102 that, in operation, extends proximally to the inferior vena cava (IVC). The arc length D refers to a length of the arc of catheter 100 that extends proximally from the physical bend of distal curve 202, through the physical bend of proximal curve 302. The proximal bend radius E of proximal curve 302 refers to the radius of a hypothetical circle that would be formed from the partial curve or arc that forms the physical curve or bend of proximal curve 302 in catheter 100. Arc length D may be about 7 mm to about 7 cm. Proximal bend radius E may be about 7 mm to about 6 cm.

The above-described ranges for arc length D and proximal bend radius E for proximal curve 302 are similar, because the two dimensions are related in that they both can be smaller, to create a tighter proximal curve 302, or both can be larger, to create a more gradual proximal curve 302, or both can be more middling within the range.

It has been realized that a catheter 100 including secondary or proximal curve 302—that is, proximal curve 302 as well as distal curve 202—may facilitate improved access to the RV, as well as improved access more centrally through the tricuspid valve, which facilitates reducing or eliminating chordae interaction, valve damage, and/or hemodynamic compromise during occluder deployment. Providing proximal curve 302 may also enhance coaxial alignment of distal portion 106 with the PDA for occluder deployment and recapture, while minimizing the amount of distortion of the PDA and the pulmonary artery.

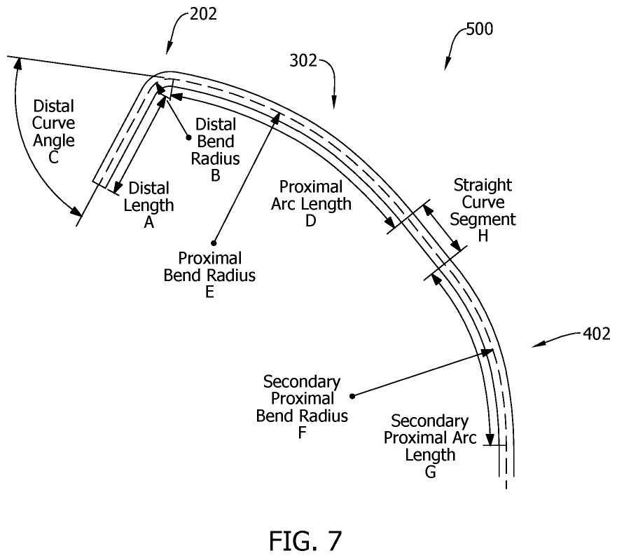

Further embodiments of a catheter (including catheter 100) also include a tertiary or “second-proximal” curve, which is proximal of the secondary or proximal curve (such as proximal curve 302). Turning to FIG. 7, an exemplary catheter 500 is depicted. Catheter 500 includes distal curved region 200 with a distal curve 202, with curve angle C, length A, and distal bend radius B; a proximal curve 302, with a proximal bend radius E and an arc length D; a second-proximal curve 402, with a second-proximal bend radius F and a second-proximal arc length G; and a straight section H along the catheter length between proximal curve 302 and the second-proximal curve 402. The second-proximal curve bend radius F may range from about 1 cm-6.5 cm, and the arc length G of the second-proximal curve 402 may range from about 2 mm-7 cm. In some embodiments, second-proximal curve 402 commences about 1.25 cm to 5 cm proximal of proximal curve 302. The commencement distance/length of the second-proximal curve 402 depends in part on the geometry of the distal curve 202, such as the radius of curvature and the arc length thereof, and/or the geometry of proximal curve 302. In some embodiments, there may be a straight section H along the catheter length between the proximal curve 302 and the second-proximal curve 402 of about 2 mm to about 1.5 cm.

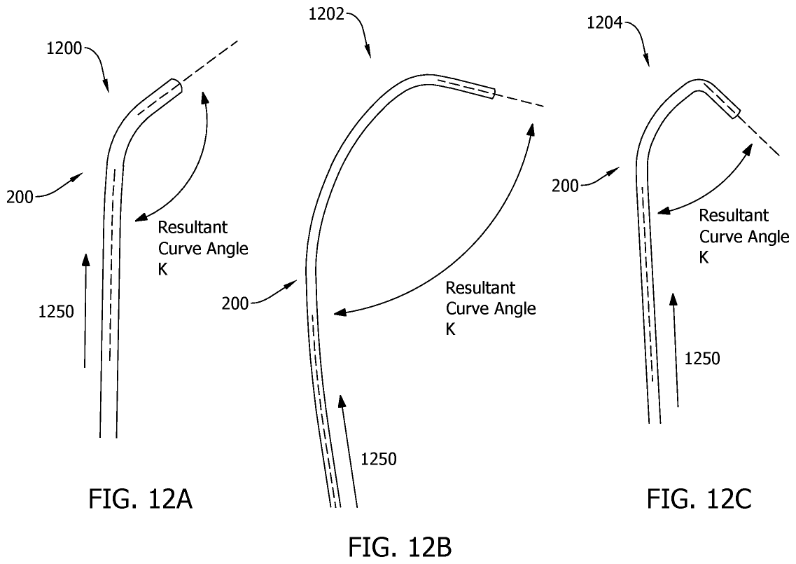

With reference now to FIGS. 12A-12C, distal curved region 200 of exemplary catheters 1200, 1202, and 1204 (each similar to catheter 100), respectively, may be described as having a resultant curve angle K that is tighter or smaller than other known guide catheters, with the combination of the distal and proximal curves 202, 302, and, in some embodiments, the combination of the distal, proximal, and second-proximal curves 202, 302, 402. The resultant curve angle K is measured between a longitudinal axis of catheter shaft 102 (at a location proximal to distal curved region 200) and a longitudinal axis taken through distal tip portion 113. A more acute resultant curve angle K results in a more downward-pointing distal tip 112.

Specifically, it has been realized in the conception of the present disclosure that with respect to delivering an occluder to a PDA, enabling distal tip 112 to point more downward, or obliquely away from a proximal-to-distal direction 1250 of the longitudinal axis of catheter shaft 102 (e.g., more clockwise, with respect to the views of FIGS. 12A-12C), may allow for right ventricle (RV) access through the tricuspid valve (TV), as distal tip 112 is able to point downward through the TV, towards the apex. The length of the overall distal curved region 200 also allows distal tip 112 to tip downward through the TV as the guidewire and catheter 100 are advanced into the anatomy.

The overall resultant curvature of distal curved region 200 (e.g., resultant curve angle K) may also reduce the potential of entanglement with any subvalvular apparatus, by allowing for more central annulus crossing. A more centered TV crossing may provide this same benefit when torquing catheter 100 in the RV into the right ventricle outflow tract (RVOT). Specifically, the shape of distal curved region 200 enables catheter 100 to torque into the RVOT without engaging—and, therefore, without damaging—the tricuspid valve chordae.

While a shorter distal length A may be advantageous for crossing into the RV, a longer distal length A may provide better alignment in the PDA for device deployment in relation to the aortic ampulla of the duct. Additionally, the above-described distal bend radius B and curve angle C of distal curve 202 may be advantageous in conjunction with proximal curve 302 for gaining access to the RV (e.g., with or without a second-proximal curve 402). Distal curve 202 is also advantageous for aligning distal tip 112 with the PDA for deployment and recapture of an occluder. Moreover, a longer distal length A of distal curve 202 may improve physician control of distal portion 106 and provide better stability in the duct.

It has also been realized that a catheter 100 including a second-proximal curve 402—as well as distal curve 202 and proximal curve 302—may further aid advancement into the RV from the RA, allowing catheter 100 to enter the RV more easily as the second-proximal curve 402 is unsheathed (e.g., by withdrawing an associated introducer). In some instances, however, a catheter having a suitably formed proximal curve 302 and no second-proximal curve 402 may function similarly to a catheter having both a proximal curve and a second-proximal curve.

In some embodiments, it may be advantageous to have a relatively shorter arc length D and a relatively smaller proximal bend radius E, to provide a catheter having a tighter proximal curve 302 and a longer, larger second-proximal curve 402. The inverse would be true as well; it may be advantageous to have a longer, larger proximal curve 302 and a smaller, shorter second-proximal curve 402. These combinations result in a resultant curve angle K that may optimize the position of distal tip 112. Because these features are related in function, the ranges for arc length D and proximal bend radius E, as provided herein, facilitate design optimization with either (i) one proximal curve 302 or (ii) a proximal curve 302 and a second-proximal curve 402.

The above-described ranges for the second-proximal curve bend radius F and second-proximal arc length G for second-proximal curve 402 are similar, because the two dimensions are related in that they both can be smaller, to create a tighter second-proximal curve 402, or both can be larger, to create a more gradual second-proximal curve 402, or both can be more middling within the range.

Additionally, in some embodiments, it may be advantageous to have a relatively short second-proximal arc length G, and a relatively small second-proximal curve bend radius F, to provide a catheter having a tighter or smaller second-proximal curve 402, when combined with a longer, larger proximal curve 302. The inverse would be true as well; it may be advantageous to have a longer, larger second-proximal curve 402 and a smaller, shorter proximal curve 302. These combinations result in a resultant curve angle K that may optimize the position of distal tip 112. Because these features are related in function, the ranges for second-proximal curve bend radius F and second-proximal arc length G, as provided herein, facilitate design optimization with either (i) one proximal curve 302, or (ii) a proximal curve 302 and a second-proximal curve 402.

It is contemplated that the overall length of distal curved region 200, including both distal curve 202 and proximal curve 302, may extend up to about 15 cm or less. A distal curved region 200 including distal curve 202, proximal curve 302, and a second-proximal curve 402, as described above, may extend up to about 15 cm or less. In some embodiments, distal curved region 200 extends up to about 10 cm or less. The length of distal curved region 200 may be minimized, to maximize torque response and pushability, but desirably has a sufficient length to ensure that, in operation of catheter shaft 102, the relatively softer distal curved region 200 (as described further herein) is located in the inferior vena cava (IVC) and through the tricuspid valve when distal portion 106 is at the aortic side of the PDA, which in turn reduces negative effects on adjacent hemodynamics and trauma caused by pressure on the adjacent anatomy.

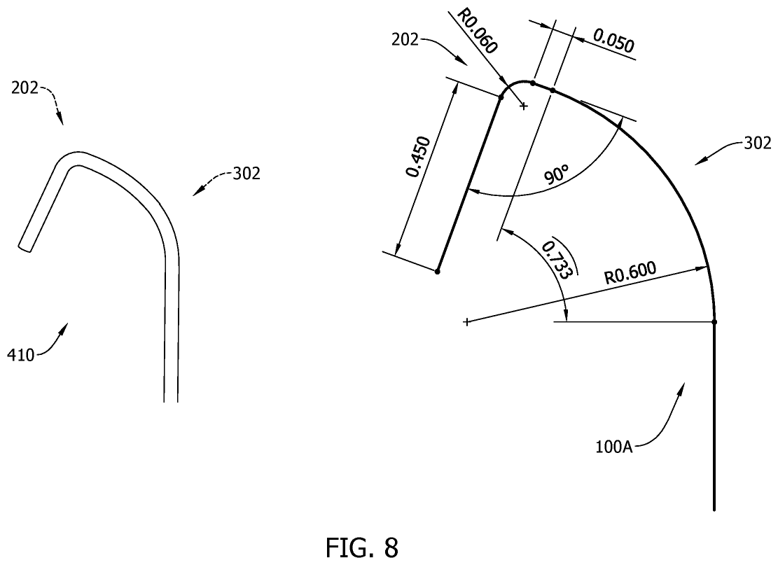

FIGS. 8-10 depict various embodiments of curve plates that may be used for forming corresponding catheters. Specifically, in each case, a catheter shaft (e.g., catheter shaft 102) may be advanced over the curve plate and heat-set to define the characteristics of the distal portion (e.g., distal portion 106) of the catheter shaft.

More specifically, FIG. 8 depicts a curve plate 410 that may be used for forming corresponding catheter 100A with a distal curved region 200 including distal curve 202 and proximal curve 302. Curve plate 410 may therefore include a portion corresponding to distal curve 202 and a portion corresponding to proximal curve 302. The resulting catheter 100A includes a distal curve 202 with a distal length A of about 12 mm (which is not specifically depicted in FIG. 8) from distal tip 112 to the middle of distal curve 202, a distal bend radius B of 0.06 inches (in or ″) (1.5-1.6 mm), and a curve angle C of 90°. Catheter 100A includes proximal curve 302 with an arc length D of about 0.7″ (17-18 mm) and a proximal bend radius E of about 0.6″ (15-16 mm).

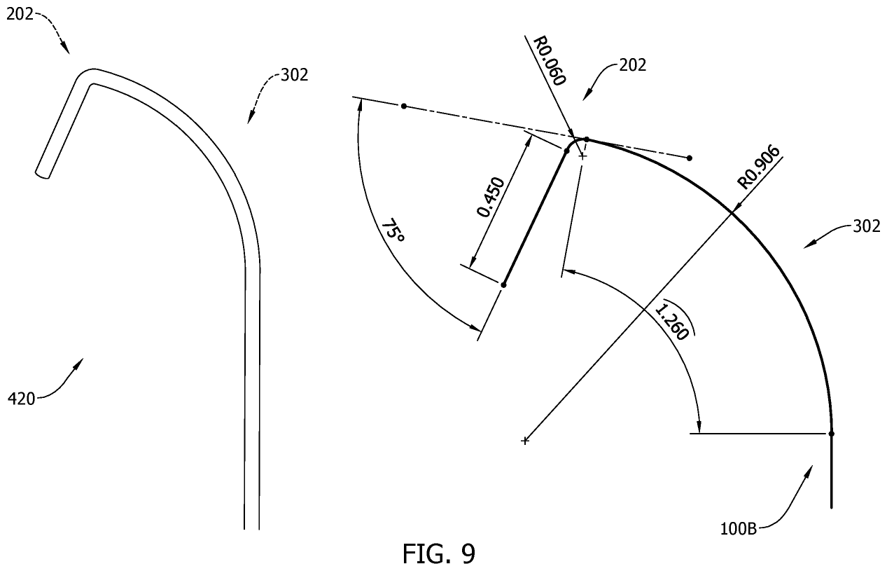

FIG. 9 depicts a curve plate 420 that may be used for forming a corresponding catheter 100B with a distal curved region 200 including distal curve 202 and proximal curve 302. Curve plate 420 may therefore include a portion corresponding to distal curve 202 and a portion corresponding to proximal curve 302. The resulting catheter 100B includes a distal curve 202 with a distal length A of about 12 mm (which is not specifically depicted in FIG. 9) from distal tip 112 to the middle of distal curve 202, a distal bend radius B of 0.06″(1.5-1.6 mm) and a curve angle C of 75°. Catheter 100B includes proximal curve 302 having an arc length D of about 1.3″ (33-34 mm) and a proximal bend radius E of about 0.9″ (22-23 mm).

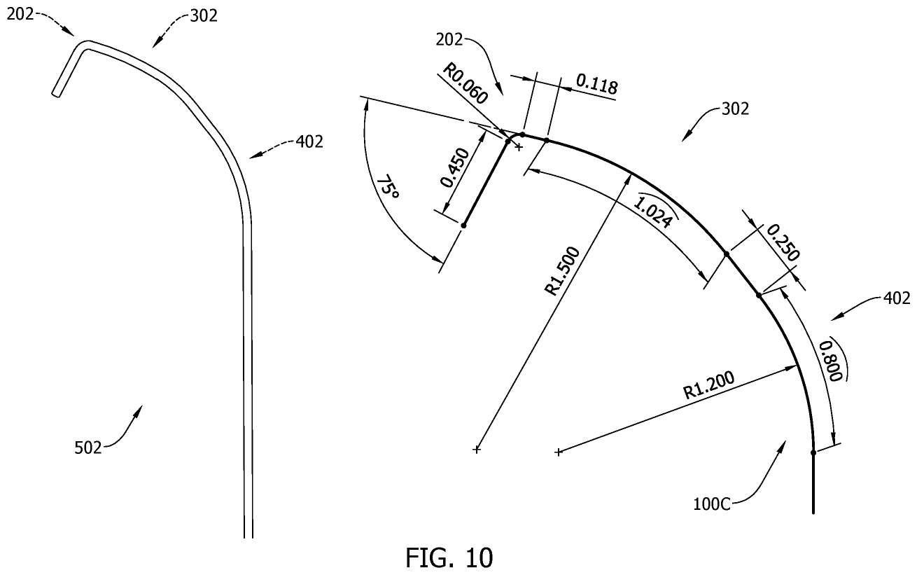

FIG. 10 depicts a curve plate 502 that may be used for forming a corresponding catheter 100C with a distal curved region 200 including distal curve 202, proximal curve 302, and second-proximal curve 402. Curve plate 502 may therefore include a portion corresponding to distal curve 202, a portion corresponding to proximal curve 302, and a portion corresponding to second-proximal curve 402. In the illustrated embodiment, catheter 100C includes distal curve 202 having a length A of about 12 mm (which is not specifically depicted in FIG. 10) from distal tip 112 to the middle of distal curve 202, a distal bend radius B of 0.06″ (1.5-1.6 mm), and a curve angle C of 75°. Catheter 100C includes proximal curve 302 having a proximal bend radius E of about 1.5″ (38-39 mm) and an arc length D of about 1.02″ (25-26 mm). Catheter 100C includes second-proximal curve 402 having a bend radius F of about 1.2″ (30-31 mm) and an arc length G of about 0.8″ (20-21 mm). A straight section H between proximal curve 302 and second-proximal curve 402 has a length of about 0.25″ (6.35 mm).

The catheters (e.g., catheter 100) of the present disclosure are selectively designed to optimize softness and strength. In the exemplary embodiment, this balance is achieved, in part, by forming catheter 100 at least partially from a braided material including, but not limited to, stainless steel, nitinol, titanium, nylon, tungsten, aramide, Kevlar™, PEEK, PET, fiberglass, platinum, iridium, nickel-cobalt, and/or any combination thereof. The braided material is covered with a reflowed resin covering or “jacket.” Specifically, using resin jackets of varying durometers (e.g., softness) and lengths within distal curved region 200 of catheter shaft 102 can facilitate optimal balance of softness and strength for the necessary maneuverability, strength for deployment and recapture of medical device(s), pushability, and torquability, as well as being atraumatic to tissue and more easily trackable through the anatomy of the patient's vasculature. Moreover, the catheters of the present disclosure may be implemented with or without tracking over a guidewire.

Catheters 100 may have distal curved regions 200 including sections (e.g., sections I-IV) having a resin jacket that is sufficiently soft to maximize flexibility and create an atraumatic/flexible distal end portion of shaft 102 and to minimize hemodynamic compromise and damage to the tricuspid valve due to stretching or “tenting” of the valve, while being stiff enough to hold its shape to travel through and/or direct a guidewire through the tricuspid valve during advancement to the target site (e.g., the PDA). Additionally, catheter shaft 102 is constructed to hold its shape during occluder delivery and deployment, to maintain alignment with the PDA for controlled deployment and recapture without deforming the surrounding anatomy or affecting placement accuracy. In exemplary embodiments, sections I-IV have a respective softness including, but not limited to, 35 Durometer (D), 40D, 45D, or 55D, for a distal length of up to about 15 cm, or about 5 cm to about 10 cm, of shaft 102. The remainder of shaft 102 proximal to distal curved region 200—that is, section V—may be stiffer than the distal curved region 200, for example, having a softness including 63D to 72D, or an even higher durometer. In one specific embodiment, section I has a resin jacket of about 40D, section II has a resin jacket of about 40D, section III has a resin jacket of about 45D, section IV has a resin jacket of about 55D, and section V has a resin jacket of about 63D. The resin jacket of shaft 102 may be formed from any suitable reflowable thermoplastic polymer, including, but not limited to, polyether block amide or PEBA (e.g., PEBAX™), urethane, polyurethane, chronoprene, pellethane, tecoflex, tecothane, silicone, polyethylene, nylon 11, nylon 12, HDPE, LDPE, MDPE, and the like, or combinations thereof.

The following table (Table I) provides example characteristics of sections I-V:

TABLE I

| | Durometer | Length | | Durometer | Length | | Durometer | Length | | Durometer | Length | |
|---|---|---|---|---|---|---|---|---|---|---|---|---|
| | (Example | (Example | | (Example | (Example | | (Example | (Example | | (Example | (Example | |
| | One) | One) | | Two) | Two) | | Three) | Three) | | Four) | Four) | |
| I | 40 D | 1 | mm | 40 D | 1 | mm | 40 D | 1 | mm | 40 D | 1 | mm |
| II | 40 D | 4 | mm | 40 D | 4 | mm | 40 D | 14 | mm | 40 D | 4 | mm |
| III | 45 D | 10 | mm | 45 D | 40 | mm | 45 D | 100 | mm | 45 D | 70 | mm |
| IV | 55 D | 45 | mm | 55 D | 10 | mm | 55 D | 45 | mm | 55 D | 10 | mm |
| V | 63 D | 39 | cm | 63 D | 39.5 | cm | 63 D | 29 | cm | 63 D | 36.5 | cm |

In further exemplary embodiments, the characteristics of sections I-V of the catheter may be varied to achieve certain features as described herein, wherein section I, extending proximally from the distal tip 112, may have a length of about 0-3 mm and a durometer of about 40D, 45D, or 55D; section II, extending proximally from section I, may have a length of about 2 mm-20 mm and a durometer of about 40D, 45D, or 55D; section III, extending proximally from section II, may have a length of about 5 mm-150 mm and a durometer of about 40D, 45D, 55D, or 63D; section IV, extending proximally from section III, may have a length of about 5 mm-150 mm and a durometer of about 45D, 55D, or 63D; and section V, extending proximally from section IV, extends the remaining length of the catheter and may have a durometer of about 63D or 72D.

In some embodiments, distal curve 202 may include tip portion 113 (e.g., section I and/or all or part of section II) of the relatively lowest durometer, such as about 45D or lower. Tip portion 113 may extend about 1 mm to about 10 mm, proximally from distal tip 112. It is advantageous for tip portion 113 to be as soft or atraumatic as possible, to reduce deformation and damage of the tissue anatomy through the vasculature up to and including the target site. However, it is further realized that having the distalmost portion of distal curve 202 be too soft may lead to distal tip 112 not being robust enough to withstand significant maneuvering and forces (e.g., multiple recaptures of an occluder at a deployment site). Accordingly, in some embodiments, distal curve 202 includes a distal tip 112 (e.g., all or part of section I) of relatively higher durometer, such as about 45D or higher, for a length of about 0.5 mm to about 3 mm, proximally from distal tip 112, followed proximally by a softest/lowest durometer tip portion 113, for increased flexibility relative to distal tip 112.

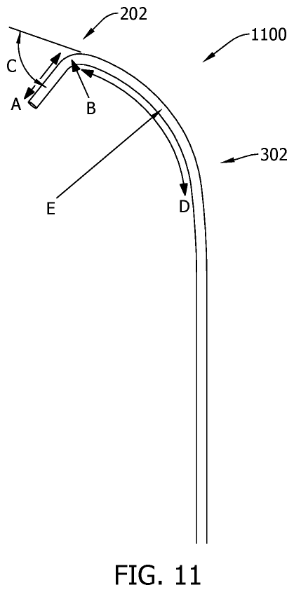

FIG. 11 depicts one exemplary catheter 100, referred to as catheter 1100, having the following characteristics. The effective length of the shaft—that is, the length of shaft 102 that extends beyond hub 110 (shown in FIG. 1), or the usable portion of shaft 102—of catheter 1100 is about 43 cm-47 cm, or about 45 cm. The extruded jacket construction of catheter 1100 has the following features, with a range around each listed value of the length of each section of about ±2 mm: (section I) 40D, 1 mm; (section II) 40D, 4 mm; (section III) 45D, 70 mm; (section IV) 55D, 10 mm; (section V) 63D, 36.5 cm. The length A of distal curve 202 is 9-11 mm, or about 10 mm. The distal bend radius B of distal curve 202 is about 3 mm-7 mm, including about 5 mm. The distal curve angle C of distal curve 202 is within about 50°-70°, or about 60°. The arc length D of proximal curve 302 is about 1 cm-5 cm, including about 3.25 cm, and the proximal bend radius E of proximal curve 302 is about 1.5 cm-5 cm or about 3 cm.

With reference again to FIG. 7, catheter 500, as described above, may have the following characteristics. The effective length of the shaft of catheter 500 is 43-47 cm, or about 45 cm. The extruded jacket construction of catheter 500 has the following features, with a range around each listed value of the length of each section of about ±2 mm: (section I) 40D, 1 mm; (section II) 40D, 4 mm; (section III) 45D, 10 mm; (section IV) 55D, 45 mm; (section V) 63D, 39 cm. The length A of distal curve 202 is 8.5-10.5 mm, or about 9.5 mm. The distal bend radius B of distal curve 202 is about 3.5 mm-8 mm, including about 6.5 mm. The distal curve angle C of distal curve 202 is within about 50°-70°, or about 60°. The arc length D of proximal curve 302 is between about 1.5 cm-5 cm, including about 2.6 cm, and the proximal bend radius E of proximal curve 302 is between about 3 cm-6 cm, including about 4.6 cm. The second-proximal bend radius F is between about 2 cm-6 cm, including about 3.6 cm, and the second-proximal arc length G is between about 1 cm-4 cm, including about 2 cm. The length of the straight segment H is between about 3 mm-1 cm, including 5 mm-7 mm, or about 6 mm.

The softness or durometer of proximal curve 302 is selected such that proximal curve 302 is sufficiently soft to maximize flexibility in the distal curved region 200, such that distal curved region 200 minimizes or eliminates anatomy distortion and tricuspid valve tenting during operation of the catheter 100. The characteristics of proximal curve 302 are also selected such that the proximal curved region is stiff enough to hold its shape to direct catheter shaft 102 and/or any guidewire through the tricuspid valve during advancement to the PDA. Also, distal curved region 200, including proximal curve 302, must hold its shape during occluder delivery and deployment to maintain alignment with the PDA, for controlled deployment and recapture without deforming the surrounding anatomy or affecting placement accuracy. In some cases, proximal curve 302 is softer (in durometer) than distal curve 202, as proximal curve 302 has a larger bend radius, and therefore is not straightened as readily during occluder advancement therethrough. In exemplary embodiments, proximal curve 302 has a softness of about 40D to about 55D.

In various embodiments, the softness or durometer of catheter shaft 102 at distal curve 202, as well as the distalmost section of distal tip portion 113, is 40D, which may facilitate providing the most flexibility and the softest tip portion 113. In other embodiments, the softness or durometer of distal curve 202, as well as the distalmost section of distal tip portion 113, is 45D, which may facilitate the above advantages while better holding the shape of distal curve 202. In some embodiments, the softness or durometer of catheter shaft 102 at proximal curve 302 is between 45D and 55D.

In operation of catheter shaft 102, section V, or the portion of catheter shaft 102 proximal of distal curved region 200, may extend (proximally of distal curved region 200) through the rest of the heart to the IVC, or may extend proximally for the remainder of catheter shaft 102. Section V is generally stiffer than the sections distal thereto, to optimize torque strength and column strength for pushability. In some embodiments, section V has a softness (durometer) of about 63D to about 72D.

Transitions in softness between adjacent sections throughout catheter shaft 102 (“durometer transitions”) may extend between about 5 mm and about 20 mm in length, to prevent drastic durometer changes that can act as kink points along catheter shaft 102. For example, in one embodiment, where proximal curve 302 has a durometer of 45D and section V (or proximal portion 104) of catheter shaft 102 has a durometer of 63D, catheter shaft 102 may include a 55D “intermediate” or “transitional” section (e.g., with a length between about 5 mm and about 20 mm).

It should be readily understood that the direction of distal curve 202 is important for the usability of catheter 100. Therefore, in the exemplary embodiment of the present disclosure, catheter 100 includes hub 110 having a visual indicator 120 thereon. In FIG. 1, this visual indicator 120 is embodied as a circle on hub 110, at an angular location corresponding to the direction of distal curve 202. In other embodiments, visual indicator 120 may be otherwise embodied, using shape(s), color(s), icon(s), tactile feature(s), and the like. In some embodiments, the visual indicator may be located elsewhere on catheter 100 that remains visible to the user thereof, such as on catheter shaft 102 (e.g., via a pad printed or laser-marked line). These visual indicators may be especially useful in echo-guided procedures, in which the curve shape and direction cannot be easily seen.

Furthermore, with respect to the construction of catheter shaft 102 internal to the above-described resin jackets, a braided construction using braided stainless steel or nitinol material may be used in many embodiments. The braid may be formed from a different material, such as titanium, nylon, tungsten, aramide, Kevlar™, PEEK, PET, fiberglass, platinum, iridium, nickel-cobalt, and/or any combination thereof. Using braided material in a catheter (e.g., catheter 100) may improve the mechanical performance thereof (e.g., column strength and torque transmission) relative to other catheters. The particular parameters of the braid can be optimized based on the particular application. For instance, torque strength and flexibility can be optimized by maximizing pics per inch (PPI) of the braid for the number of wires (or constructing the braid with less than but similar to the maximum PPI) used, and/or by adjusting the wire width and/or thickness. In some embodiments, a 16 flat wire 0.001×0.003″ (0.0254×0.0762 mm) braid may facilitate minimizing impact on a thickness of the braid wall (discussed further herein) while maintaining desired robustness of catheter shaft 102 against torque and bending forces. In other embodiments, a 16 wire 0.001×0.003″ (0.0254×0.0762 mm) flat wire braid at 55 to 80 PPI construction, in particular, 80 PPI, provides the desired characteristics. The use of flat wire for the above construction of the braid may include the benefits of increased torque strength and maximizing the wall thickness of the polymer resin jacket. In another embodiment, a 16 round wire 0.00125″ (0.03175 mm) or 0.0015″ (0.0381 mm) at maximum PPI, or less than or similar to maximum PPI, is used to construct the braid. In yet another embodiment, a 32 round wire 0.00125″ (0.03175 mm) or 0.0015″ (0.0381 mm) at maximum PPI, or less than or similar to maximum PPI, in a paired diamond braid pattern is used. The use of round wire for the above constructions may result in reduced torque strength, and higher PPI can be achieved.

In some embodiments, catheter 100 may have a braid construction that terminates proximal to distal curve 202 or, alternatively, terminates along the length A proximal to distal tip 112. In some specific embodiments, the braid construction extends through a full length of catheter shaft 102 until the location of a markerband (as described further herein). In such cases, the polymer jacket would feature an increase in thickness (where there is no braid) to maintain an outer diameter (OD) and an inner diameter (ID), and create a soft, unbraided distal end of catheter 100. A polymer jacket on the distal section of catheter 100 (e.g., on distal portion 113) would allow the catheter to be as soft as possible. If the braid terminates just proximal to distal curve 202, so the curved portion is unbraided, catheter 100 may exhibit better curve retention of distal curve 202. To achieve the benefit, while also maintaining strength, it may be beneficial for the braid to terminate as distally as possible, yet proximal to distal curve 202. The braid may terminate between 10-25 mm from distal tip 112. In one embodiment, with a catheter 100 having a length A of 9 mm and a distal bend radius B of 4.5 mm, an unbraided distal length may have the braid terminate 15 mm from distal tip 112 of catheter 100. In another embodiment, an unbraided distal section may be made up of a thicker 45D section for 15 mm proximal from distal tip 112, a thinner 45D section once the braid begins for an additional 60 mm, and a transition of 55D for 10 mm to 63D for the remainder of the proximal catheter. In yet another embodiment, a thicker 55D section may form the distal unbraided section of catheter 100, for 15 mm proximal from distal tip 112, a thinner 55D section once the braid begins for an additional 60 mm, and 63D for the remainder of the proximal portion of catheter 100. Notably, however, a lack of braid in distal curve 202 may reduce the kink resistance and torque strength of distal portion 106 of shaft 102. Accordingly, at least some preferred embodiments include the braided material through distal portion 106, including throughout distal curve 202.

However, it is also understood that coils can, in some instances, offer improved performance in flexibility and kink resistance, compared to braids. Therefore, it is realized herein that utilizing both coil and braid construction in a single catheter shaft can facilitate optimizing catheter performance, such as where certain column strength, axial strength (e.g., resistance to elongation), and torque transmission requirements must be maintained, while also providing optimal kink resistance and flexibility to navigate through small anatomies and maximize the atraumatic behavior of distal tip 112, ensuring catheter shaft 102 flexes in response to contacting tissue. In one or more embodiments of the present disclosure, distal curved region 200 includes a coil (not shown) at distal tip 112. This coil may extend about 1 mm to about 15 mm proximally from distal tip 112, or through all/part of section I, all or part of section II, and/or all or part of section III, or distal thereto, and may provide enhanced performance for PDA access and PDA occluder deliverability and recapture, as well as a more flexible, atraumatic tip (compared to braided construction alone).

Additionally, consideration must be made toward the profile of catheter shaft 102. In particular, the OD of shaft 102 must be small enough to suitably navigate a patient's vasculature (the importance of which is enhanced for young and/or small patients), and the ID must be suitably sized to accommodate the occluder to be deployed at the target site. In some embodiments, the ID must additionally or alternatively accommodate a guidewire during the initial navigation phase.

For example, it may be advantageous to have a smaller ID adjacent to distal tip 112, to reduce any mismatch or difference between the shaft ID and the guidewire OD. This improved sizing may facilitate reducing a potential of tissue damage through the vasculature up to and including the target site. Moreover, decreasing the ID of shaft 102 at distal tip portion 113 while maintaining a consistent shaft OD may provide a relatively thicker shaft wall at distal tip portion 113, in turn providing a blunter and more atraumatic tip.

It may also be advantageous for catheter shaft 102 to have a more “standard” ID proximal to distal tip 112 (e.g., proximal of distal curved region 200). For example, having a shaft ID of about 0.045″ (1.143 mm) or about 0.046″ (1.168 mm) may be beneficial to maintain standard use forces of catheter 100 and reduce resistance to flow, for contrast injection implementations.

Accordingly, in certain exemplary embodiments, the ID of catheter shaft 102 is varied along the length thereof. In particular, the ID of shaft 102 tapers (decreases) distally along shaft 102. It is contemplated that a standard guidewire OD is about 0.035″ (0.889 mm), so it would be beneficial to minimize the catheter ID at distal tip portion 113 as close as possible to the guidewire OD, such as about 0.036″ (0.914 mm) to about 0.038″ (0.965 mm). However, it is also realized that the catheter ID is also limited by a maximum profile of the medical device (e.g., occluder) to be delivered therethrough. Therefore, in at least one exemplary embodiment, a proximal catheter ID of about 0.045″ (1.143 mm) or 0.046″ (1.168 mm), tapered down to a distal catheter ID of about 0.0415″ (1.054 mm) to about 0.044″ (1.112 mm) (e.g., about 0.0435″ (1.105 mm) or 0.0425″ (1.080 mm)), may provide an optimal balance of the above-described advantages. Notably, it is further contemplated that the catheter ID at the distalmost end of distal tip portion 113 (e.g., the distalmost 0.5 mm-3 mm) may be even further reduced, where the flexibility of distal tip portion 113 is enhanced to enable expansion of distal tip portion 113 during deployment of the medical device. Additionally or alternatively, where a maximum profile of the medical device is reduced, a catheter ID of about 0.036″ (0.914 mm) to about 0.038″ (0.965 mm) may be readily usable.

The taper may be implemented in a transition or taper region that is a distance of about 3 mm to about 40 mm, measured proximally from distal tip 112. This distance may be selected to optimize one or more characteristics of catheter shaft 102. For example, a longer distance from distal tip 112 to the taper region allows for a thicker extrusion (e.g., of the resin jacket) to be used at distal curved region 200 (e.g., at distal curve 202), which can be advantageous for curve retention in this region. Conversely, a shorter distance from distal tip 112 to the taper region may increase contrast flow while minimizing a force required to inject the contrast; this shorter distance may also minimize wall thickness near the distal curve 202, and, therefore, increase the flexibility of distal tip portion 113 and make distal tip 112 more atraumatic. The shorter distance may also reduce the force of the medical device going through the distal curvature. The length of the tapered region may be about 2 mm-4 mm, or about 3 mm. In some embodiments, the ID of shaft 102 is selectively varied, during formation of shaft 102, using a mandrel with a (complementary) varying or tapering outer diameter. In one particular embodiment, shaft 102 has a proximal catheter ID of about 0.045″ (1.143 mm), which is tapered down at about 8 mm from distal tip 112 for a length of about 3 mm, to achieve a distal tip ID of 0.0435″ (1.105 mm). Such an embodiment may provide a reduced diameter mismatch while maintaining necessary use forces.

In one or more embodiments, catheter shaft 102 further includes a hydrophilic coating or lubricious additive to the resin jacket, to reduce the friction coefficient of the outer surface of the resin jacket. In some embodiments, having a more lubricious surface of shaft 102 for the distal 25 cm of shaft 102 is advantageous to reduce the friction of catheter 100 through an introducer valve. If a hydrophilic coating is used, it may be advantageous to minimize the length of shaft 102 that includes the coating—to include the length of shaft 102 that is advanced through the introducer valve—such that proximal portion 104 is not slippery in a physician's gloves. When utilizing a lubricious additive, the entire length of shaft 102 may have the additive in the resin jacket without compromising usability. A lubricious additive may be most beneficial in lower durometer resins, which would be advantageous with respect to the catheter 100 of the present disclosure because the softest (low durometer) resins are implemented nearest to distal tip 112.

In some embodiments, catheter shaft 102 also includes a markerband (not shown), surrounding a portion of distal tip portion 113 for imaging visibility during the cardiac procedure. The markerband may be a platinum/iridium (Pt/Ir) markerband that is disposed around the distal end of the braid and reflowed into the resin jacket. In some embodiments, the markerband may be formed from an alternative material, such as gold, titanium, tungsten, tantalum, and/or combinations thereof.

Notably, however, such a markerband must be disposed a short distance from the distal tip 112, to ensure that the markerband is fully encapsulated by the resin. In one example embodiment, the markerband is at least about 0.001″ from distal tip 112, or, more particularly, about 1 mm (±0.5 mm) from distal tip 112. In some instances, distal tip portion 113 includes an amount of material, such as a polymer or relatively soft resin, that extends distally past the markerband. This material allows for some radial compliance at distal tip 112, which reduces the forces required to deploy and recapture any medical device (e.g., occluder) delivered using catheter 100.

However, due to the space taken up by the markerband with the standard construction, the resin jacket is thinner in the areas adjacent to the markerband, which reduces the tensile strength of those areas adjacent to distal tip 112. Therefore, in other exemplary embodiments, radiopaque materials may be added to the resin during the reflow process. Radiopaque materials include, but are not limited to, tungsten, bismuth, barium, titanium, and zirconium oxide. In these embodiments, the radiopaque material additive provides for a radiopaque marker that can extend fully to distal tip 112, which may enhance usability of catheter shaft 102. Additionally, the use of the radiopaque material as an additive eliminates the areas of reduced tensile strength, thereby providing a relatively more robust distal tip portion 113 compared to standard construction. In some embodiments, the radiopaque material (e.g., tungsten) is provided as an additive of about 30% to about 80% by weight of the resin, including about 50% by weight.

Moreover, in various exemplary embodiments, catheter shaft 102 itself is made to be echogenic, or visible under ultrasound, to improve the navigation of catheter 100 to the target site for optimal deployment of the occluder thereat. In some embodiments, glass microspheres are embedded in the resin jacket of shaft 102. The size of the microspheres, the percent by volume of the microspheres in the resin, and the length of distal portion 106 formed to include the microspheres may all be selected to create an optimal visible catheter shaft 102 under ultrasound. For example, the size of the microspheres may be selected based on visibility, while minimizing any effect on the surface roughness or the mechanical properties of catheter shaft 102. Glass microspheres may range in size from about 15 microns to about 60 microns. The percent by volume of resin including the microspheres may be varied based on the optimal resultant visualization under echo, while minimizing any disruption to the mechanical performance of the resin jacket. Glass microspheres may be embedded at a percent by volume of about 3% to about 30%, or, in particular, about 10% to about 20%. The portion of catheter shaft 102 including microspheres may be optimized such that catheter shaft 102 does not obscure the PDA occluder under echo. This portion may therefore be positioned about 1 mm to about 5 mm, or about 2 mm, proximally from distal tip 112. In some cases, it may be advantageous to reduce this portion as much as possible while still keeping the portion visible. In some embodiments, a similar effect may be achieved using a thin coating of an echogenic material on distal tip portion 113 (e.g., an echogenic material printed or sprayed onto distal tip portion 113).

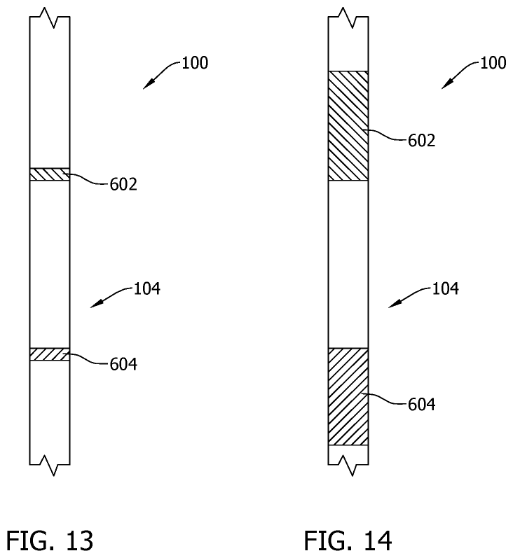

In some embodiments, catheter 100 includes at least one of first and second markers 602, 604 thereon, as depicted in FIGS. 13 and 14. More specifically, in operation, users of catheter 100 (e.g., physicians) are interested in knowing the length of catheter 100 that has been advanced into the patient's anatomy. The user does not want the catheter to advance too far into the atrium, which may increase the risk of tissue damage. Accordingly, a first marker 602 is provided on proximal portion 104 of catheter 100, which indicates to the user of catheter 100 that catheter 100 has been advanced some known first length into the patient's anatomy. First marker 602 has a first appearance (e.g., color) and its (longitudinal) placement is selected to correspond to this known first length of advancement. First marker 602 may be printed, etched, laser marked, or otherwise provided on the outer surface of catheter 100.

Additionally, the user of catheter 100 is also interested in knowing, once catheter 100 is advanced through the PDA, that catheter 100 is not advanced too much further, or too deep into the descending aorta, which can increase the risk of tissue damage. Accordingly, a second marker 604 is provided on proximal portion 104 of catheter 100, which indicates to the user of catheter 100 that catheter 100 has been advanced some known second length into the patient's anatomy. Second marker 604 is located proximal to first marker 602 and may have a second appearance (e.g., color) that is advantageously different from the first appearance of first marker 602, such that second marker 604 is easily distinguished from first marker 602. The (longitudinal) placement of second marker 604 is selected to correspond to the known second length of advancement. Second marker 604 may be printed, etched, laser marked, or otherwise provided on the outer surface of catheter 100.

First and second markers 602, 604 may be relatively thin markers, as shown in FIG. 13. In other embodiments, as depicted in FIG. 14, first and second markers 602, 604 may have an extended length, corresponding to a range of acceptable known lengths for navigation of catheter 100 through the patient's anatomy. For instance, a first (distal) end of a marker may correspond to smaller patients (e.g., a relatively shorter known length), and the second (proximal) end of the marker may correspond to larger patients (e.g., a relatively longer known length). Additionally, in some embodiments, first and second markers 602, 604 have labels or other markings that identify which of the first and second markers 602, 604 is which and/or the known length represented by the corresponding marker. First and second markers 602, 604 are particularly advantageous in echo-guided procedures, in which the location of distal tip 112 is not identified by fluoroscopy.

In some embodiments, catheter assembly 100 is implemented as part of a delivery system compatible with smaller guidewires, which may include a dilator. For example, a guidewire may be inserted through the dilator, and the dilator and guidewire guided to the target site. The dilator would bridge the “gap” between the small guidewire OD and the catheter ID, as described above, to reduce or eliminate mismatch, and would further aid in tracking to the target site by tracking using a softer, less traumatic dilator before advancing the catheter over the dilator. The dilator may be formed from coil and/or braided construction and/or may include a resin jacket with a soft durometer. The dilator has sufficient softness to easily track over the flexible guidewire to the descending aorta, and, during removal, to limit impact on the position of the catheter (e.g., catheter 100) deployed over the dilator at the end of the PDA. A dilator reveal length—that is, a length of the dilator that extends past the distal end of the catheter—at or between 1 mm to 20 cm may be beneficial, depending on the particular application. Having a longer dilator at or between 1 cm to 20 cm longer than catheter 100 may be beneficial to allow traversing the anatomy with the dilator past the PDA first, before following with the catheter.

Although a number of embodiments of this disclosure have been described above with a certain degree of particularity, those skilled in the art could make numerous alterations to the disclosed embodiments without departing from the spirit or scope of the disclosure. All directional references (e.g., upper, lower, upward, downward, left, right, leftward, rightward, top, bottom, above, below, vertical, horizontal, clockwise, and counterclockwise) are only used for identification purposes to aid the reader's understanding of the present disclosure, and do not create limitations, particularly as to the position, orientation, or use of the disclosure. Joinder references (e.g., attached, coupled, connected, and the like) are to be construed broadly and may include intermediate members between a connection of elements and relative movement between elements. As such, joinder references do not necessarily infer that two elements are directly connected and in fixed relation to each other. It is intended that all matter contained in the above description or shown in the accompanying drawings shall be interpreted as illustrative only and not limiting. Changes in detail or structure may be made without departing from the spirit of the disclosure as defined in the appended claims.

Any patent, publication, or other disclosure material, in whole or in part, that is said to be incorporated by reference herein is incorporated herein only to the extent that the incorporated materials does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

What is claimed is:

1. A catheter comprising a shaft formed from braided material encapsulated by a resin jacket, wherein the catheter shaft extends from a proximal portion to a distal tip, the catheter shaft comprising a distal curved section including an acute distal curve proximal of the distal tip.

2. The catheter of claim 1, comprising a 3-5 French (Fr) catheter.

3. The catheter of claim 1, wherein the resin jacket has a varying durometer along a length thereof.

4. The catheter of claim 3, wherein the resin jacket has a lower durometer along the distal curved section and a higher durometer proximal of the distal curved section.

5. The catheter of claim 1, wherein the distal tip is rounded, thick, blunt, and/or soft, due at least in part to the resin jacket.

6. The catheter of claim 1, wherein a portion of the resin jacket including or adjacent to the distal tip is echogenic.

7. The catheter of claim 1, wherein the distal curved section also includes a proximal curve proximal of the distal curve.

8. The catheter of claim 7, wherein the distal curved section has a resultant curve angle that directs the distal tip downward and obliquely away from a proximal-to-distal direction of a longitudinal axis of the shaft.

9. The catheter of claim 7, wherein the proximal curve has a larger bend radius and arc length than a bend radius and arc length of the acute distal curve.

10. The catheter of claim 9, wherein the distal curved section further includes a second-proximal curve proximal of the proximal curve.

11. The catheter of claim 10, wherein the second-proximal curve is spaced from the proximal curve by a straight section.

12. The catheter of claim 1, wherein the catheter shaft has an inner diameter that decreases towards the distal tip.

13. The catheter of claim 1, wherein the catheter shaft further comprises a coil section distal of the braided material at the distal tip.

14. The catheter of claim 1, further comprising (i) a hydrophilic coating on the resin jacket, or (ii) a lubricious additive in the resin jacket.

15. The catheter of claim 1, further comprising a radiopaque markerband adjacent to the distal tip.

16. The catheter of claim 1, wherein a portion of the resin jacket including or adjacent to the distal tip comprises a radiopaque material additive that acts as a radiopaque marker.

17. The catheter of claim 1, further comprising a hub adjacent to the proximal portion.

18. The catheter of claim 17, wherein the hub comprises a visual indicator aligned with a direction of the distal curve.

19. The catheter of claim 1, further comprising a marker on the proximal portion thereof.

20. A medical device delivery and recapture system comprising:
the catheter of claim 1; and
a dilator.
